(19) United States
(12) Patent Application Publication
(10) Pub. No.: US 2017/0036147 A1
(43) Pub. Date: Feb. 9, 2017

(54) FILTERS COMPRISING MICROBIALLY-PRODUCED CELLULOSE

(71) Applicant: Uzair Nadeem Mohammad, Livermore, CA (US)

(72) Inventor: Uzair Nadeem Mohammad, Livermore, CA (US)

(21) Appl. No.: 15/302,904

(22) PCT Filed: Apr. 6, 2015
(86) PCT No.: PCT/US15/24572

§ 371 Date: Oct. 7, 2016

Related U.S. Application Data
(60) Provisional application No. 61/976,708, filed on Apr. 8, 2014.

(51) Int. Cl. B01D 39/18; B01D 29/00; C12P 19/04

(52) U.S. Cl. CPC B01D 39/18; C12P 19/04; B01D 29/0093; B01D 2239/0216

(57) ABSTRACT

A fluid filter formed of microbially-produced cellulose, preferably formed into a web of cellulose fibers in artificial culture of the microbes, then removed from the culture and shaped into a filter. The shaping may include a compression step and may also include an embossing step. Methods of culturing and forming the filter are also disclosed.

### CROSS REFERENCE TO RELATED APPLICATIONS

The present application claims the benefit of priority to U.S. Provisional Patent Application No. 61/976,708 filed Apr. 8, 2014. The entire disclosure of the aforementioned application is expressly incorporated by reference in its entirety.

### BACKGROUND OF THE INVENTION

Field of the Invention

The present disclosure relates to microbially-produced cellulose filter materials and methods for making the same.

Description of the Related Art

Filters find widespread application in consumer, agricultural, laboratory, automotive, utility, water treatment, commercial and industrial processes. The cost of filter material can be substantial, particularly the cost of filters capable of removing relatively small particles from a fluid.

A wide variety of natural and synthetic materials have been used to produce filter materials. These include various natural fibers and natural materials formed into fibers, such as those made of keratin, wood fiber, cotton, wool, silk, flax hemp, latex, and glass. A large number of synthetic polymers have also been used to form filters, such as polyester, nylon, silicone, aramid, polyacetal, and the like.

The process of manufacturing a filter, such as one made of wood fiber, is relatively complex. In one exemplary process, for example, wood is harvested and then cut into chips. The chips normally first enter presteaming zone where they are wetted and preheated with steam. Cavities inside fresh wood chips are partly filled with liquid and partly with air. The steam treatment causes the air to expand and about 25% of the air to be expelled from the chips. The next step is to impregnate the chips with sodium sulfide and sodium hydroxide (white liquor) and a lignin solution (black liquor). This begins a chemical reaction that facilitates separation of lignin in the wood from the cellulose fibers.

After an incubation period at warm temperature, the wood chips are cooked digesters for several hours at 170 to 176° C. (338 to 349° F.). Under these conditions lignin and hemicellulose degrade to give fragments that are soluble in the strongly basic liquid. The resulting solid pulp is collected.

After the fibers have been separated, the mill washes and decontaminates the pulp. To produce a white filter material, the mill must bleach the pulp to remove color associated with remaining residual lignin. Typically, the bleaching chemicals (such as chlorine dioxide, oxygen, or hydrogen peroxide) are injected into the pulp and the resulting mixture is washed with water.

The bleached or unbleached wood pulp is then pumped onto vibrating wire screen mats to allow water to drain out of the pulp and to help the fibers interlock into sheets. Resulting fibers are then pressed into sheets with optional binder materials, and are then formed into the final filter shape.

It would be advantageous to provide a much simpler process for making cellulose-based filter material.

### SUMMARY OF THE INVENTION

One embodiment disclosed herein is a fluid filter, comprising a web of interlinked cellulose fibers, secreted by microorganisms grown in artificial culture to form said web, wherein said web has been shaped into a fluid filter subsequent to secretion. Preferably, the filter further comprises a reinforcing structure embedded in the web. Advantageously, the filter has been formed by compressing the web of cellulose fibers that was secreted by bacteria. In an embodiment, the cellulose fibers are those secreted by Glucanoaceterbacter xylinus.

Also disclosed is a method for making a filter, comprising culturing cellulose-secreting microorganisms in a culture system that provides a surface to induce secretion of microbial cellulose fibers in the form of a web at the surface, removing the web of cellulose fibers from the surface, and shaping the web to provide a filter.

The method may also advantageously include providing a reinforcing structure on or adjacent to the surface during secretion of said fibers, such that said reinforcing structure becomes embedded in said fibers.

In one embodiment, the method also includes comprising compressing the secreted fibers, such as compression to reduce the thickness of the web by at least about 10, 20, 30, 40, 50, 60, 70, 80, or 90 percent. The compressing process can also advantageously include embossing a texture onto the filter.

In any of the embodiments, the surface in the culture system may be a solid surface, or alternatively may comprise a top surface of a liquid culture medium at a liquid-gas interface.

In some embodiments, the microorganisms are of the genera Acetobacter, Glucanoaceterbacter Sarcina ventriculi or Agrobacterium. In a preferred embodiment, the microorganism is Glucanoaceterbacter xylinus. In some embodiments the microorganisms are selected from Acetobacter pasteurianum, Acetobacter rancens, Acetobacter xylinum, Sarcina ventriculi, and Bacterium xylinoides. In other embodiments, the microorganisms are of the genera Phaetophyta, Rhodophyta, or Chrystophyta. In yet other embodiments, the microorganisms are algae or fungi.

Still another embodiment is a genetically modified unicellular organism, comprising one or more heterologous genes coding for production of cellulose, wherein the genes are CesA genes.

In some embodiments, a fluid filter is provided, wherein the fluid filter comprises a web of interlinked cellulose fibers, secreted by bacteria grown in artificial culture to form said web, wherein said web has been shaped into a fluid filter subsequent to said secretion. In some embodiments, the fluid filter further comprises a reinforcing structure embedded in the web. In some embodiments, the filter is formed by compressing the web of cellulose fibers that was secreted by bacteria. In some embodiments, the cellulose fibers are those secreted by Glucanoaceterbacter xylinus.

In some embodiments, a method for making a filter is provided, wherein the method comprises culturing cellulose-secreting microorganisms in a culture system that provides a surface to induce secretion of microbial cellulose fibers in the form of a web at the surface and removing the web of cellulose fibers from the surface and shaping the web to provide a filter. In some embodiments, the method further comprises providing a reinforcing structure on or adjacent to said surface during secretion of said fibers, such that said reinforcing structure becomes embedded in said fibers. In some embodiments, the method further comprises compressing the secreted fibers. In some embodiments, the method further comprises embossing a texture onto the filter. In some embodiments, the surface is a solid surface. In some embodiments, the surface is a top surface of a liquid culture medium at a liquid-gas interface. In some embodiments, the microorganisms are of the genera Acetobacter, Glucanoaceterbacter Sarcina ventriculi or Agrobacterium. In some embodiments, the microorganism is Glucanoaceterbacter xylinus. In some embodiments, the microorganisms are selected from Acetobacter pasteurianum, Acetobacter rancens, Acetobacter xylinum, Sarcina ventriculi, and Bacterium xylinoides. In some embodiments, the microorganisms are of the genera Phaetophyta, Rhodophyta, or Chrystophyta. In some embodiments, the microorganisms are algae. In some embodiments, the microorganisms are fungi. In some embodiments, the culture system comprises a carbon source. In some embodiments, the carbon source comprises Glucose, Sucrose, Glycerol, Ethanol and/or Mannitol. In some embodiments, the carbon source is Mannitol. In some embodiments, the culture system comprises a pH of 5.0, 5.1, 5.2, 5.3, 5.4, 5.5, 5.6, or any other pH between any two values listed. In some embodiments, the culture system comprises a pH of 5.5. In some embodiments, the method further comprises adjusting the pH of the culture system by adding an acid. In some embodiments, the acid is citric acid, apple cider vinegar, or vinegar.

In some embodiments, a genetically modified unicellular organism is provided, wherein the genetically modified unicellular organism comprises one or more heterologous genes coding for production of cellulose, wherein the genes are CesA genes.

In some embodiments, a method for making a filter comprises culturing cellulose-secreting microorganisms in a culture system that provides a surface to induce secretion of microbial cellulose fibers in the form of a web at the surface and removing the web of cellulose fibers from the surface and shaping the web to provide a filter. In some embodiments, the method further comprises deconstructing the cellulose fibers by blending. In some embodiments, the blending is performed by a rapidly spinning blade, for example such as found in a food processer or a blender, to generate a blended mixture. In some embodiments, the blended mixture is hydrated with water and/or NaOH to neutralize the microorganism and treat the cellulose fibers. In some embodiments, the method further comprises air drying, compressing, or freezing to reform the cellulose fibers into a filter.

### DETAILED DESCRIPTION

The present disclosure is based on the discovery that cellulose-producing microorganisms can be induced to deposit high-purity cellulose fibers to form a web or sheet having predetermined shape or configuration to form excellent filter material that requires minimal post-deposition processing. The microorganisms can typically be grown in a number of culture media or carbon-containing feedstocks to minimize production costs.

Microbial cellulose is somewhat different from plant cellulose, and has greater strength, higher purity, better moldability, and increased hydrophilicitiy. It has no lignin or hemicellulose. Tensile strength is higher. The structure is more crystalline. Importantly, the microbial cellulose fibers are significantly smaller than those of plant origin, typically <0.1 μm versus about 10 μm for wood pulp fibers—more than two orders of magnitude difference. All of these properties combine to produce filters having higher mechanical strength and much smaller pore size than conventional cellulose filters derived from plant fiber.

A number of cellulose-secreting microorganisms are known. Bacterial cellulose is produced, for example, by certain bacteria of the genera Acetobacter, Glucanoaceterbacter Sarcina ventriculi and Agrobacterium. Examples of cellulose-secreting bacteria include Acetobacter pasteurianum, Acetobacter rancens, Acetobacter xylinum, Glucanoaceterbacter xylinus, Sarcina ventriculi, and Bacterium xylinoides. A suitable strain of G. xylinus is available from the American Type Culture collection as ATCC Strain 53582. Certain algae, such as Phaetophyta, Rhodophyta, and Chrystophyta produce cellulose, as do some fungi where cellulose forms a layer inside the cell wall.

Genetically-engineered organisms are also contemplated herein as a source of microbial cellulose. The CesA pathway, and/or the genes bcsA bcsB bcsC bcsD are known genetic elements that encode the pathway for cellulose production in G. xylinus. Production of cellulose by insertion of the cellulose-production pathway into other bacteria, yeast, or other organisms can provide an alternative and potentially optimized expression system for microbial cellulose.

In the production of filter materials, a desired microbial strain capable of producing the desired cellulose is cultured in a suitable growth medium. In general, any medium providing a carbon source, preferably a sugar, as well as oxygen, essential minerals, and other elements known to facilitate growth of the particular microorganism. For cellulose production, a culture pH of between about 5 and 8 is preferred. While glucose and other processed sugars can be used, it is also possible to use crude sugar sources or waste streams from various industrial or agricultural processes that contain sugars to provide a low cost growth medium. Exemplary growth media include, for example,
LB Broth Media (powdered standard LB (Lysogeny broth) mixed with water).
Tea Culture
   For every 500 mL of water, 1 green tea and 1 black tea bag, steeped for 2 minutes.
A mixture of LB broth media and Tea Culture
   ¾ LB and ¼ Tea by volume is one suitable ratio.
G. Xylinus specific media (ATCC specified)
   Per 1 Liter of water: 20 g Glucose, 5 g Peptone, 5 g Yeast Extract, 2.7 g Disodium Phosphate, 1.5 g Citric Acid.
      This is the general formula for optimal cellulose production; in other words, the cells require these basic nutrients to produce cellulose. Normal LB broth does produce cellulose, but LB broth does not contain enough of these nutrients for optimal cellulose production. Other media, such as those made from fruits or tea, are essentially just natural sources for these same nutrients.
Date Syrup
   200 g seedless date fruit blended into 500 mL water, filtered with cheese cloth, and diluted to a final date concentration of 20%

In each instance, it is preferred that the growth media be sterilized prior to use to remove any undesired microorganisms. Heat sterilization, radiation sterilization, UV sterilization, chemical sterilization, or any other suitable sterilization technique may be used.

In some embodiments, a carbon source for the microbe is added. In some embodiments the carbon source is added to the culture system. Carbon sources can include but is not limited to Glucose, Sucrose, Glycerol, Ethanol and other alcohols, and Mannitol. Mannitol appears to be the most efficient source of carbon for bacteria. In some embodiments, the carbon source comprises Glucose, Sucrose, Glycerol, Ethanol and other alcohols, and/or Mannitol. In some embodiments, the carbon source is Mannitol. Carbon sources for the microbes can be found in Ruka et al (Altering the growth conditions of Gluconacetobacter xylinus to maximize the yield of bacterial cellulose; Carbohydrate Polymer, June 20; 89(2):613-622; incorporated by reference in its entirety herein)

Exemplary growth media can be balanced for their pH. The pH in growth media affects the rate of cellulose production. A pH closer to 4 favors cell propagation, while a pH closer to 5.5 is more ideal for cellulose production. The pH can be balanced by addition of citric acid to carbon-source containing media. Vinegars, like Apple Cider Vinegar, or other acetic acid or other organic acid source also can be used to create an ideal pH environment, and may introduce other factors that encourage growth of cellulose-producing bacteria. In some embodiments of the growth media for the microbes, the pH of the growth media is balanced. In some embodiments the pH is balanced by the addition of citric acid. In some embodiments the pH is balanced by the addition of vinegar. In some embodiments the pH is balanced by the addition of Apple Cider Vinegar. In some embodiments, the pH of the growth media is balanced to a pH of 5.0. In some embodiments, the pH of the growth media is balanced to a pH of 5.1. In some embodiments, the pH of the growth media is balanced to a pH of 5.2. In some embodiments, the pH of the growth media is balanced to a pH of 5.3. In some embodiments, the pH of the growth media is balanced to a pH of 5.4. In some embodiments, the pH of the growth media is balanced to a pH of 5.5. In some embodiments, the pH of the growth media is balanced to a pH of 5.6.

A completed filter can be constructed by deconstructing the grown webs of cellulose by blending them (in a food processor, blender, etc). The blended material, hydrated with a bit of water and potentially with NaOH or other chemical treatments that can neutralize the bacteria present, or treat the fibers. The blended mixture can be poured into a desired shape, either alone or atop a filter substrate, like filter paper or fabric. The blended material then can be air dried, compressed, or frozen to allow the blended cellulose to reform into a usable web, now reshaped. In some embodiments, the filter is constructed by deconstructing the grown webs of cellulose by blending them (in a food processor, blender, etc) into a blended mixture. In some embodiments, the blended mixture is hydrated with water and/or NaOH that can neutralize the bacteria present, or treat the fibers. In some embodiments, the blended mixture is air dried, compressed, or frozen to allow the blended cellulose to reform into a usable web, now reshaped.

This reshaping allows for inconsistencies present in the grown cellulose webs to be eliminated (holes, thinner portions, or rips/tears) by blending, adding consistency and manufacturing flexibility.

The blended material can be formed into more than a disk filter shape (the unblended cellulose filters are essentially restricted to a disk shape). For instance, the blended material can be poured into a cylinder to make a thick tube shaped filter if a hole is bored through the center of the cylinder.

In some embodiments, a method for making a filter is provided, wherein the method comprises culturing cellulose-secreting microorganisms in a culture system that provides a surface to induce secretion of microbial cellulose fibers in the form of a web at the surface and removing the web of cellulose fibers from the surface and shaping the web to provide a filter. In some embodiments of the method, the filter is constructed by deconstructing the grown webs of cellulose by blending them. In some embodiments, the blending is performed by a food processor to generate a blended mixture. In some embodiments, the blending is performed by a blender to generate a blended mixture. In some embodiments, the blended mixture is hydrated with water. In some embodiments, the blended mixture is further mixed with NaOH or other chemical treatments that can neutralize the bacteria present, or treat the fibers. In some embodiments, the blended mixture is poured into a desired shape, either alone or atop a filter substrate, like filter paper or fabric. In some embodiments, the blended mixture is air dried, compressed, or frozen to allow the blended cellulose to reform into a usable web, now reshaped. In some embodiments, the microorganisms are of the genera Acetobacter, Glucanoaceterbacter Sarcina ventriculi or Agrobacterium.

### EXAMPLE 1

### Preparation of Bacterial Culture

Freeze-dried pellets of G. Xylinus (ATCC Strain 53582) were dissolved and resuspended in 2 mL of LB Broth. After resuspension, the 2 mL was added to a larger quantity of media, in this case 1 liter, and was maintained between about 22 Celsius and 37 Celsius, ideally at about 30 Celsius for 1 to 2 days to allow stock culture to grow. The stock culture was then maintained under refrigeration until ready for use. Other volumes of medium can be innoculated from this original stock.

### EXAMPLE 2

### Preparation of Filter Media from Bacterial Culture

The following process was used for the production of a prototype bacterial cellulose filter through use of the following exemplary steps:

1. Media Preparation: Although any of the media disclosed herein could be used, LB Broth was selected for this example. 25 g of powdered LB broth was dissolved in 1 L water, stirred, and was sterilized by autoclave for 30 minutes OR microwaved until boiling, cooled, and stirred again.

2. The LB Broth medium was poured into a 10″×10″ sterilized tray, and then covered with plastic wrap to maintain sterility and avoid contamination.

3. 2 mL of G. Xylinus culture prepared in Example 1 was added to the tray by lifting a corner of plastic and pipetting culture into the media within the tray. (Culture preparation methods below)

4. The tray was again securely covered with plastic wrap to ensure an air-tight seal and to maintain sterility. The plastic was perforated with a needle across its entire surface to allow air movement in and out of the tray, and a fibrous cellulose barrier was used to cover the plastic to prevent airborne bacteria or contaminates from falling through perforations while still allowing air exchange. (Of course, other methods for oxygen introduction and gas exchange can be used to prevent bacterial contamination.)

5. In approx. 4 days, a cellulose mat approximately 1 inch thick grew on top of the media.

6. The cellulose mat was removed from the top of the tray with gloved hands, and was placed in another 10″×10″ tray. The removed mat was then sterilized with 70% ethanol in the second tray by partially submerging the mat in ethanol and then shaking the tray to expose the entire mat to ethanol.

7. The first tray from which the mat was removed was maintained in a sterile environment and several subsequent mats were grown. Typically, this amount of medium should be sufficient to produce 3 or 4 mats.

8. After sterilizing in alcohol for at least about 3 minutes, the cellulose mat was washed with water to remove the alcohol, and then dried by blotting and evaporation.

9. The dried cellulose mat was then compressed between two solid surfaces.

Sufficient pressure was applied to reduce the thickness by about a factor of 5; e.g., it was compressed until it was about 0.5 cm thick.
a. Note that modifications can be made to the compression process to embosss the cellulose mat. Specifically, one or both of the compressing surfaces can be textured, such as using a heavy-weave fabric on one or both sides during compression or having a compressing surface molded or machined to a desired extent to provide a dimpled pattern, a cross-hatched pattern, or any other desired texture.

10. The compressed filter was then tested by interposing it in a liquid flow path to demonstrate its ability to remove fine particles from the liquid stream, with excellent removal of particles larger than 1 micron.

11. For disposal, the cellulose filter can be composted in organic waste bins, or sterilized if it is known to have filtered potentially harmful bacteria, and then composted.

### EXAMPLE 3

### Preparation of Date Fruit Growth Medium

A bacterial growth medium was prepared from low-cost natural material using sub-par date fruits. In particular, 200 g of stone free, low quality date fruits were combined with 500 ml of distilled water, then mixed in a blender for 1 min at low speed, and for an additional 3 min at a higher speed. The homogenized extract was filtered through a double layer of cheese cloth. The residue was then washed with hot water and solution made up to the volume required to make a concentration of 20%, following the technique of Nasab, et at., Iranian J. Biotech. 9: no. 2, 94-101 (2011), which is incorporated herein by reference in its entirety for its teaching of preparation of growth media and growth of cellulose-producing bacteria.

Although the present disclosure has been made with reference to various exemplary embodiments, it is intended that the scope of the present patent should be determined by reference to the appended claims, and not limited to any particular exemplary embodiment.

1. A fluid filter, comprising:
a web of interlinked cellulose fibers, secreted by cellulose-secreting microorganisms, wherein the cellulose-secreting microorganisms are grown in artificial culture to form said web, wherein said web has been shaped into a fluid filter subsequent to said secretion, wherein the cellulose-secreting microorganisms comprises fungi, algae or bacteria, and wherein the cellulose-secreting microorganisms are genetically modified.

2-29. (canceled)

30. The filter of claim 1, further comprising a reinforcing structure embedded in the web.

31. The filter of claim 1, formed by compressing the web of cellulose fibers that was secreted by the cellulose-secreting microorganisms.

32. The filter of claim 1, wherein the cellulose fibers are those secreted by Glucanoaceterbacter xylinus.

33. The filter of claim 1, wherein the cellulose-secreting microorganisms are genetically modified by insertion of a cellulose production pathway.

34. The filter of claim 1, wherein the cellulose production pathway comprises a CesA pathway genes, bcsA, bcsB, bcsC and/or bcsD.

35. The filter of claim 1, wherein the algae is Phaetophyta, Rhodophyta or Crystophyta.

36. A method for making a filter, comprising:
culturing cellulose-secreting microorganisms in a culture system that provides a surface to induce secretion of microbial cellulose fibers in the form of a web at the surface, wherein the cellulose-secreting microorganisms comprises fungi, algae or bacteria and wherein the cellulose-secreting microorganisms are genetically modified; and
removing the web of cellulose fibers from the surface and shaping the web to provide a filter.

37. The method of claim 36, further comprising providing a reinforcing structure on or adjacent to said surface during secretion of said fibers, such that said reinforcing structure becomes embedded in said fibers.

38. The method of claim 36, further comprising compressing the secreted fibers.

39. The method of claim 38, further comprising embossing a texture onto the filter.

40. The method of claim 36, wherein the surface is a solid surface.

41. The method of claim 36, wherein the surface is a top surface of a liquid culture medium at a liquid-gas interface.

42. The method of claim 36, wherein the cellulose-secreting microorganisms are of the genera Acetobacter, Glucanoaceterbacter Sarcina ventriculi or Agrobacterium.

43. The method of claim 42, wherein the cellulose-secreting microorganisms is Glucanoaceterbacter xylinus.

44. The method of claim 36, wherein the cellulose-secreting microorganisms are selected from Acetobacter pasteurianum, Acetobacter rancens, Acetobacter xylinum, Sarcina ventriculi, and Bacterium xylinoides.

45. The method of claim 36, wherein the cellulose-secreting microorganisms are algae.

46. The method of claim 36, wherein the cellulose-secreting microorganisms are fungi.

47. The method of claim 46, wherein the fungi are of the genera Phaetophyta, Rhodophyta, or Chrystophyta.

48. The method of claim 36, wherein the culture system comprises a carbon source.

49. The method of claim 48, wherein the carbon source comprises Glucose, Sucrose, Glycerol, Ethanol and/or Mannitol.

50. The method of claim 49, wherein the carbon source is Mannitol.

51. The method of claim 36, wherein the culture system comprises a pH of 5.0, 5.1, 5.2, 5.3, 5.4, 5.5, 5.6, or any other pH between any two values listed.

52. The method of claim 51, wherein the culture system comprises a pH of 5.5.

53. The method of claim 36, wherein the method further comprises adjusting the pH of the culture system by adding an acid.

54. The method of claim 53, wherein the acid is citric acid, apple cider vinegar, or vinegar.

55. The method of claim 36, wherein the cellulose-secreting microorganisms are genetically engineered for expression for microbial cellulose by insertion of a cellulose production pathway.

56. The method of claim 55, wherein wherein the cellulose production pathway comprises a CesA pathway genes and/or genes bcsA, bcsB, bcsC and/or bcsD.

57. A genetically modified unicellular organism, comprising one or more heterologous genes coding for production of cellulose, wherein the genes are CesA, bcsA, bcsB, bcsC and/or bcsD.

58. A method for making a filter, comprising:
culturing cellulose-secreting microorganisms in a culture system that provides a surface to induce secretion of microbial cellulose fibers in the form of a web at the surface; and
removing the web of cellulose fibers from the surface and shaping the web to provide a filter, wherein the cellulose-secreting microorganisms are fungi, algae or bacteria, and wherein the fungi, algae or bacteria is genetically modified.

59. The method of claim 58, wherein the method further comprises deconstructing the cellulose fibers by blending.

60. The method of claim 59, wherein the blending is performed by a food processer or a blender to generate a blended mixture.

61. The method of claim 60, wherein the blended mixture is hydrated with water and/or NaOH to neutralize the microorganism and treat the cellulose fibers.

62. The method of claim 61, further comprising air drying, compressing, or freezing to reform the cellulose fibers into a filter.

63. The method of claim 58, wherein the fungi are of the genera Phaetophyta, Rhodophyta, or Chrystophyta.
